(19) United States
(12) Patent Application Publication
(10) Pub. No.: US 2018/0271403 A1
(43) Pub. Date: Sep. 27, 2018

(54) BREATH SENSOR

(71) Applicants: NGK SPARK PLUG CO., LTD., Aichi (JP); SPIROSURE, INC., Pleasanton, CA (US)

(72) Inventors: Keizo FURUSAKI, Nagoya-shi (JP); Masatoshi UEKI, Niwa-gun (JP); Kenji NISHIO, Komaki-shi (JP); Ryan LEARD, Oakland, CA (US); Solomon SSENYANGE, Fremont, CA (US)

(73) Assignees: NGK SPARK PLUG CO., LTD., Aichi (JP); SPIROSURE, INC., Pleasanton, CA (US)

(21) Appl. No.: 15/468,547

(22) Filed: Mar. 24, 2017

(51) Int. Cl. A61B 5/08; G01N 33/00; G01N 33/497; G01N 27/16; G01N 27/407; G01N 27/406; H05B 3/02; H05B 3/06; H05B 3/12; H05B 3/16

(52) U.S. Cl. CPC A61B 5/082; G01N 33/0037; G01N 33/497; G01N 27/16; G01N 27/4074; G01N 27/4076; A61B 2010/0087; H05B 3/026; H05B 3/06; H05B 3/12; H05B 3/16; G01N 2033/4975; G01N 27/4067

(57) ABSTRACT

A breath sensor (1) includes a heat exchange portion (41) that allows heat exchange between breath discharged from a second chamber C2 and breath introduced into a first chamber C1. Therefore, breath introduced into the first chamber C1 can be heated by breath discharged from the second chamber C2, to increase the temperature of the introduced breath. Since the temperature of the breath is increased, an effect of reducing power consumption of a heater (29c) in heating a conversion portion (21) and a detection portion (29a) is realized. The heater (29c) heats both the conversion portion (21) and the detection portion (29a) to a temperature in an operation or activation temperature range. Further, power consumption for heating to an operation temperature can be reduced by preheating the introduced breath as described above.

### BACKGROUND OF THE INVENTION

### 1. Field of the Invention

The present disclosure relates to a breath sensor that detects, for example, a concentration of a specific component present in breath.

### 2. Description of the Related Art

To date, for example, a sensor that measures an extremely low concentration (several ppb to several hundred ppb level) of NOₓ in breath for the diagnosis of asthma, has been known (see Patent Document 1).

The sensor is configured as one unit in which a conversion portion having a catalyst that includes PtY (zeolite having Pt supported thereon) for converting NO in breath to NO₂, and a detection portion having a mixed-potential type sensor element that detects NO₂ are combined.

In the sensor, a temperature at which the catalyst optimally acts and a temperature at which the sensor element optimally operates are different. Therefore, the conversion portion has a heater for heating the catalyst, and the detection portion has a heater for heating the sensor element, and the heaters are controlled so as to be separately set to different temperatures.

[Patent Document 1] US Patent Application Publication No. 2015/0250408

### 3. Problems to be Solved by the Invention

However, in the above-described conventional art, the sensor has two heaters, and the two heaters are controlled so as to be separately set to different temperatures. Therefore, a problem arises in that wasted heat that is dissipated from each heater is increased to increase power consumption of the heaters, or it is difficult to make the sensor compact.

In order to address the problem, a sensor in which a conversion portion and a detection portion are heated by a single heater may be considered. Specifically, as illustrated in FIG. 9, a sensor P11 may be considered. In the sensor P11, a sensor body portion P8 into which an adjustment unit P3 having a conversion portion P2 in a first chamber P1, a sensor unit P6 having a detection portion P5 in a second chamber P4, and a single heater P7 that heats the conversion portion P2 and the detection portion P5 are integrated, is accommodated in a housing P9. Further, in the sensor P11, a gas flow pipe P10 that connects the first chamber P1 and the second chamber P4 extends so as to pass through the outside of the housing P9.

In the sensor P11, breath (G) can be introduced from the outside of the housing P9 through a breath introduction pipe P12 into the first chamber P1, introduced from the first chamber P1 through the gas flow pipe P10 into the second chamber P4, and discharged from the second chamber P4 through a breath discharge pipe P13 to the outside of the housing P9.

In the sensor P11, for example, the heater P7, the conversion portion P2, and the detection portion P5 are arranged so that the temperature of the conversion portion P2 and the temperature the detection portion P5 can be independently adjusted. For example, when the heater P7 is disposed closer to the detection portion P5 than to the conversion portion P2, the temperature of the conversion portion P2 and the temperature of the detection portion P5 can be set so as to be different from each other.

However, in the sensor P11 using the single heater P7, breath at a normal temperature is supplied to a catalyst in the conversion portion P2 which operates at a high temperature (for example, 200° C. to 300° C.), and a problem thus arises in that the temperature of the catalyst is lowered, and the efficiency of the catalyst is reduced.

In order to address this problem, breath introduced from the outside into the first chamber may be preheated by a different heater. However, in this case, a problem of increased power consumption as in the case of two heaters being used as described above, is not solved.

### SUMMARY OF THE INVENTION

The present invention has been made in view of the aforementioned circumstances, and an object thereof is to provide a breath sensor that can operate with low power consumption by effectively utilizing heat.

The above object has been achieved by providing, in accordance with a first aspect (1) of the invention, a breath sensor which comprises an adjustment unit having a first chamber into which breath is introduced, and having a conversion portion that converts, to a second gas component, a first gas component included in the breath that is introduced into the first chamber; a sensor unit having a second chamber into which the breath that has passed through the adjustment unit is introduced, and having a detection portion having an electric characteristic which varies with a change in concentration of the second gas component; a single heater configured to heat the conversion portion and the detection portion; and a gas flow path configured to connect the first chamber and the second chamber in a state in which at least a part of the gas flow path extends outside the adjustment unit and outside the sensor unit.

Furthermore, the adjustment unit, the sensor unit, and the heater are integrated into a sensor body portion in a state where the adjustment unit and the heater are thermally coupled to each other, and the sensor unit and the heater are thermally coupled to each other.

Moreover, the breath sensor comprises a housing which surrounds an outer circumference of the sensor body portion. Also, a heat exchange portion that allows for heat exchange between breath discharged from the second chamber and breath introduced into the first chamber is provided in at least the housing.

The breath sensor according to the first aspect (1) includes a heat exchange portion that allows heat exchange between the breath discharged from the second chamber and the breath introduced into the first chamber. Therefore, breath (that is, breath which has been heated by the heater and which has a high temperature, and, hereinafter, also referred to as discharged breath) discharged from the second chamber can be used to heat and thereby increase the temperature of breath (that is, breath which is exhaled from a human body and has a temperature lower than a temperature of breath heated by the heater, and, hereinafter, also referred to as introduced breath) introduced into the first chamber.

Since the temperature of the introduced breath which is introduced into the first chamber of the adjustment unit is increased (that is, the introduced breath can be preheated), power consumption of the heater in heating the conversion portion and the detection portion can be reduced. That is, the heater heats both the conversion portion and the detection portion to a temperature within an operation temperature range, and, if the introduced breath can be preheated, power consumption for heating to an operation temperature can be reduced.

Thus, according to the first aspect (1), since heat can be effectively utilized, an effect of reducing power consumption can be significantly realized.

In particular, in a case where the breath sensor is incorporated in a compact potable device, power consumption of a power supply for heating the heater can be reduced, and the effect thereof is thus significant.

In a preferred embodiment (2), the breath sensor (1) above further comprises a chamber opening through which breath is discharged from the second chamber into the housing, a housing opening through which the breath in the housing is discharged to an outside of the housing, and a breath introduction pipe that passes through the housing opening, connects an inside of the first chamber and the outside of the housing, and allows the breath to be introduced into the first chamber from the outside of the housing, may be provided.

According to the breath sensor (2), a breath introduction pipe is provided that allows breath to be introduced from the outside of the housing into the first chamber. Therefore, heat exchange between the introduced breath in the breath introduction pipe and the discharged breath (for example, discharged breath at the circumference of the breath introduction pipe in the housing) on the outer circumferential side of the breath introduction pipe can be efficiently performed. Thus, power consumption of the heater can be further reduced.

In a preferred embodiment (3), the breath sensor (2) above further comprises a breath discharge pipe provided on an outer surface of the housing that allows the breath to be discharged from an inside of the housing to an outside of the housing. Further, the breath discharge pipe has a through hole that is in communication with the housing opening, and is disposed so as to surround the entirety of a circumference of the housing opening. Also, the breath introduction pipe is disposed so as to pass through the through hole of the breath discharge pipe.

According to the breath sensor (3), the breath introduction pipe is disposed so as to pass through the through hole (that is, the inside) of the breath discharge pipe, whereby heat exchange between the introduced breath in the breath introduction pipe and the discharged breath in the breath discharge pipe (that is, on the outer circumferential side of the breath introduction pipe) can be efficiently performed. Thus, power consumption of the heater can be further reduced.

In yet another preferred embodiment (4), the breath sensor (1) above further comprises a breath introduction pipe that extends from an outside of the housing through the second chamber into the first chamber, and a breath discharge pipe that extends from an inside of the second chamber to an outside of the housing. The breath introduction pipe is disposed so as to pass through a through hole of the breath discharge pipe.

According to the breath sensor (4), the breath introduction pipe is disposed so as to extend from the outside of the housing through the second chamber into the first chamber such that the breath introduction pipe extends through the through hole of the breath discharge pipe that extends from the inside of the second chamber up to the outside of the housing. Thus, heat exchange between: the introduced breath in the breath introduction pipe; breath, in the breath discharge pipe, discharged from the second chamber; and the breath inside the second chamber, can be efficiently performed. Thus, power consumption of the heater can be further reduced.

### BRIEF DESCRIPTION OF THE DRAWINGS

FIG. 1 is a plan view of a breath sensor according to a first embodiment.

FIG. 2 is a cross-sectional view showing a cross-section (A-A cross-portion in FIG. 1) of the breath sensor according to the first embodiment.

FIG. 3 is a cross-sectional view showing an enlarged cross-section (A-A cross-section in FIG. 1) of a sensor body portion according to the first embodiment.

FIG. 4 is a cross-sectional view showing a cross-section (B-B cross-section in FIG. 1) of the breath sensor according to the first embodiment.

FIG. 5 is a cross-sectional view of a breath sensor according to a second embodiment in a state where the breath sensor is cut along an inlet or the like.

FIG. 6 is a cross-sectional view of a breath sensor according to a third embodiment in a state where the breath sensor is cut along an inlet or the like.

FIG. 7 is a cross-sectional view showing, in an enlarged manner, a part of the breath sensor according to the third embodiment in a state where the breath sensor is cut along a breath discharge pipe.

FIG. 8 is a cross-sectional view of a breath sensor according to another embodiment in a state where the breath sensor is cut along an inlet or the like.

FIG. 9 is a perspective view of a breath sensor obtained by improving a conventional art in a state where the breath sensor is cut along an inlet or the like.

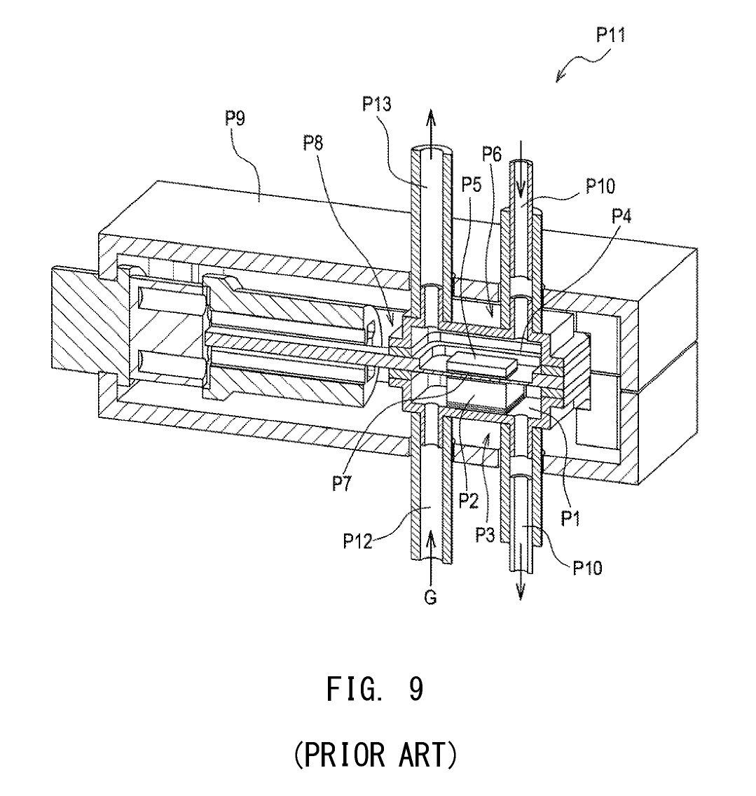

### DESCRIPTION OF REFERENCE NUMERALS

Reference numerals used to identify various features in the drawings include the following.

1, 101, 201: breath sensor; 3: housing; 5: adjustment unit; 7: sensor unit; 13, 301: gas flow pipe; 21: conversion portion; 22, 33, 203: inlet; 23, 35: outlet; 29a: detection portion; 29c: heater; 37: sensor body portion; 39: housing opening; 41, 107, 211: heat exchange portion; 103, 205: breath discharge pipe; 105, 207: through hole; C1: first chamber; C2: second chamber

### DETAILED DESCRIPTION OF THE PREFERRED EMBODIMENTS

Hereinafter, embodiments of a breath sensor to which the present disclosure is applied, will be described with reference to the drawings. However, the present invention should not be construed as being limited thereto.

### 1. First Embodiment

### [1-1. Overall Structure of Breath Sensor]

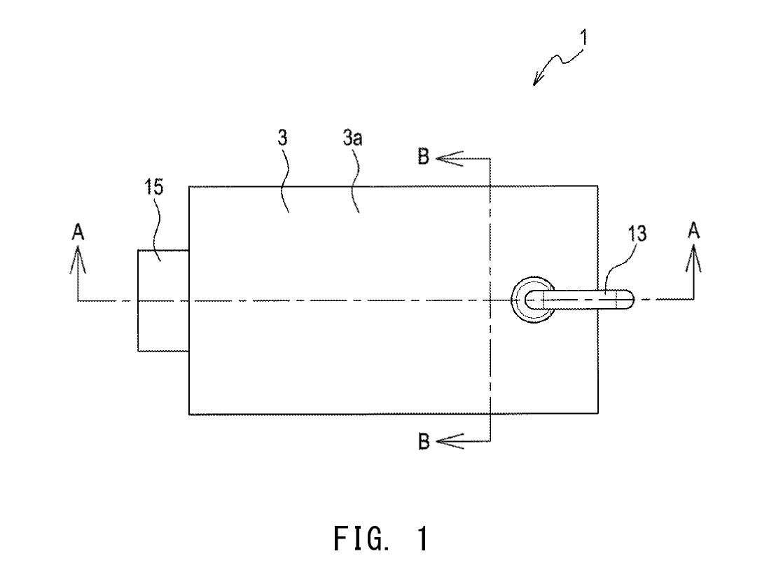

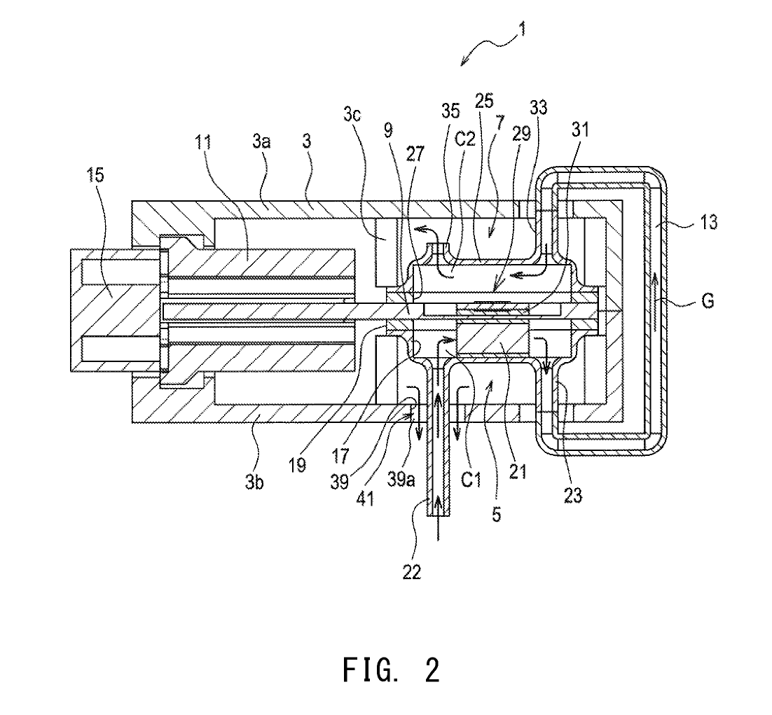

As shown in FIG. 1 and FIG. 2, a breath sensor 1 of a first embodiment has an adjustment unit 5, a sensor unit 7, a ceramic wiring substrate 9, and a first connector portion 11 accommodated in a housing 3. Further, the breath sensor 1 includes a gas flow pipe 13 that connects the adjustment unit 5 and the sensor unit 7, and a second connector portion 15 connected to the first connector portion 11. These will be described in detail below.

As shown in FIG. 1, the housing 3 is an almost rectangular-parallelepiped-shaped casing, and is formed from, for example, a heat-resistant resin such as PPS (polyphenylene sulfide), PSU (polysulfone), or PPSU (polyphenylsulfone).

The housing 3 has a structure in which a pair of cases 3a, 3b each of which is almost rectangular-box-shaped and has an opening on one side, are combined from the upper side and the lower side shown in FIG. 2 such that the openings oppose each other, as shown in FIG. 2.

As shown in FIG. 2, the adjustment unit 5 has: a case 17 which is almost rectangular-box-shaped, has a flange, has an open upper surface (opens upward in FIG. 2), and is made of a metal; a sealing member (packing) 19 which is formed from a rectangular-frame-shaped mica and comes into contact with the flange of the case 17; a conversion portion 21 accommodated in the case 17; and the ceramic wiring substrate 9.

The flange of the case 17 contacts the lower surface of the sealing member 19, and the outer circumferential portion of the lower surface of the ceramic wiring substrate 9 contacts the upper surface of the sealing member 19, whereby the opening of the case 17 is closed by the ceramic wiring substrate 9. A first chamber C1 is formed as an internal space inside the closed case 17.

An inlet (that is, breath introduction pipe) 22 and an outlet 23 that are pipe-shaped and serve as piping connection ports are formed so as to be spaced from each other and project from the lower surface of the case 17. Further, the inlet 22 and the outlet 23 are in communication with the first chamber C1.

The conversion portion 21 that is porous and which can transmit gas therethrough is disposed between the inlet 22 and the outlet 23 in the first chamber C1. The conversion portion 21 is structured so as to convert a first gas component (for example, NO) included in breath, to a second gas component (for example, NO₂), as described below.

In the adjustment unit 5, the breath (G) introduced through the inlet 22 into the first chamber C1 contacts the conversion portion 21 and has its gas component converted, and is thereafter discharged through the outlet 23 into the gas flow pipe 13.

The sensor unit 7 has: a case 25 which is almost rectangular-box shaped, has a flange, has an open lower surface, and is made of a metal; a sealing member 27 which is formed from a rectangular-frame-shaped mica and is adhered to the flange of the case 25; a sensor element portion 29 accommodated in the case 25; an adhesive layer 31; and the ceramic wiring substrate 9.

The flange of the case 25 contacts the upper surface of the sealing member 27, and the outer circumferential portion of the upper surface of the ceramic wiring substrate 9 contacts the lower surface of the sealing member 27, whereby the opening of the case 25 is closed by the ceramic wiring substrate 9. A second chamber C2 is formed as an internal space inside the closed case 25.

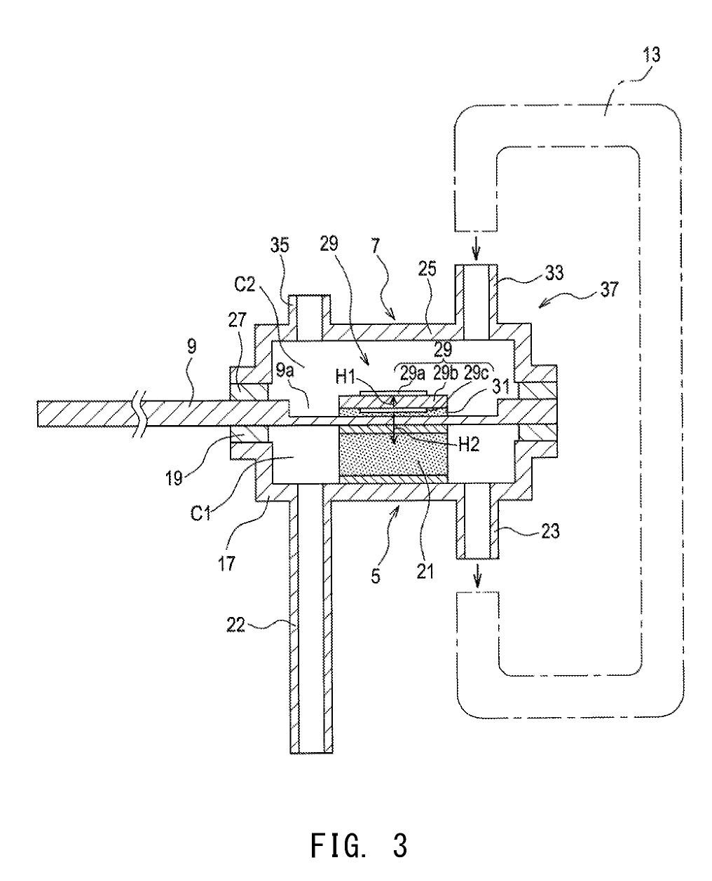

As shown in FIG. 3, the sensor element portion 29 is almost rectangular-plate-shaped. In the sensor element portion 29, a detection portion 29a is disposed on the upper surface (upper portion in FIG. 3) of a base portion 29b, and a heater 29c is disposed on the lower surface of the base portion 29b. That is, the sensor element portion 29 has a laminated structure in which the detection portion 29a, the base portion 29b, and the heater 29c are integrally layered.

Among them, the detection portion 29a forms a known mixed-potential type detection portion having a solid electrolyte body and a pair of electrodes, and has an electric characteristic which varies with a change in concentration of the second gas component, as described below (see, for example, U.S. Patent Application Publication No. US 2015/0250408 incorporated herein by reference in its entirety). The base portion 29b is a ceramic substrate which is made of, for example, alumina, and has electrical insulation properties. The heater 29c heats the detection portion 29a to an operation temperature by passing electric current therethrough, and is formed from, for example, a resistance heating element that is made of platinum, tungsten, or the like and that is formed on the surface of a ceramic substrate. The detection portion 29a may be a detection portion formed from a metal oxide semiconductor instead of the mixed-potential type detection portion.

A recess 9a (see FIG. 3) is formed at the center of the upper surface of the ceramic wiring substrate 9, and the sensor element portion 29 is disposed through the adhesive layer 31 on the bottom surface of the recess 9a such that the heater 29c faces the bottom surface of the recess 9a.

Further, pipe-shaped inlet 33 and outlet 35 are formed so as to be spaced from each other and project from the upper surface of the case 25. Also, the inlet 33 and the outlet 35 are in communication with the second chamber C2.

The sensor element portion 29 is disposed between the inlet 33 and the outlet 35 in the second chamber C2 as viewed along the longitudinal direction of the ceramic wiring substrate 9, and disposed on the recess 9a of the ceramic wiring substrate 9.

The gas flow pipe 13 is made of, for example, a resin or a metal. As shown in FIG. 2, one end of the gas flow pipe 13 is connected to the outlet 23 of the first chamber C1 and the other end of the gas flow pipe 13 is connected to the inlet 33 of the second chamber C2. That is, the first chamber C1 and the second chamber C2 are in communication with each other so as to allow breath to flow through the gas flow pipe 13.

One end portion and the other end portion of the gas flow pipe 13 are disposed in the housing 3, while the other portions thereof are disposed outside the housing 3 along the outer circumferential surface of the housing 3.

On the upper and lower surfaces, including the end portions (on the left side in FIG. 2), of the ceramic wiring substrate 9, a wiring pattern connected to the detection portion 29a and a wiring pattern that is electrically connected to the heater 29c are formed, which is not shown. These wiring patterns are connected to a metal terminal (not shown) disposed in the first connector portion 11, and the metal terminal is connected to an external circuit connecting lead wire (not shown) disposed in the second connector portion 15.

As shown in FIG. 3, the sensor unit 7 and the heater 29c are thermally coupled to each other as indicated by an arrow H1 by the heater 29c being layered through the detection portion 29a and the base portion 29b in the sensor unit 7.

Similarly, the adjustment unit 5 and the heater 29c are thermally coupled to each other as indicated by an arrow H2 by the heater 29c being layered through the conversion portion 21 in the adjustment unit 5, a part of the ceramic wiring substrate 9, and the adhesive layer 31.

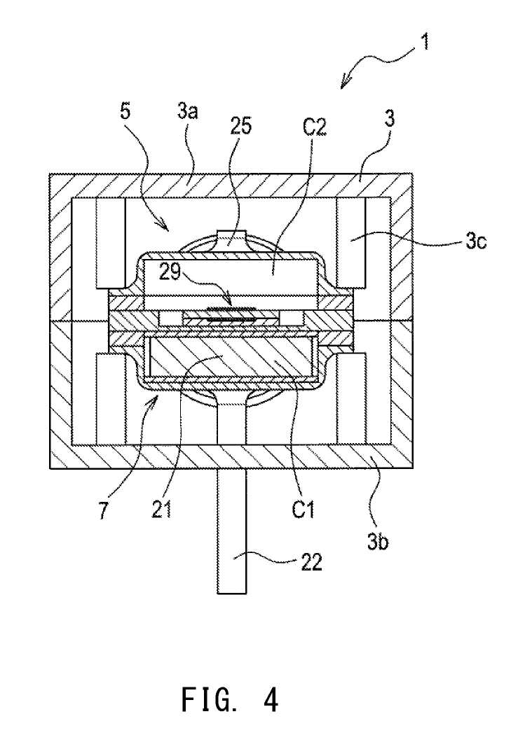

A sensor body portion 37 is configured such that the adjustment unit 5, the sensor unit 7, and the heater 29c are integrated with each other. The sensor body portion 37 is fixed in the housing 3 by means of a plurality of locking portions 3c (see FIG. 4) that project into the housing 3.

That is, in the sensor body portion 37, the above-described thermal coupling allows the single heater 29c to heat the conversion portion 21 of the adjustment unit 5 and the detection portion 29a of the sensor unit 7.

The phrase “the sensor unit 7 and the heater 29c are thermally coupled to each other” means that a member that forms the sensor unit 7 and the heater 29c are directly coupled to each other so as not to contain air therebetween and thereby enable thermal conduction. The phrase “the adjustment unit 5 and the heater 29c are thermally coupled to each other” also describes a similar state.

### [1-2. Flow Path of Breath]

Next, a flow path of breath in the breath sensor 1 will be described.

As shown in FIG. 2, the outlet (that is, chamber opening) 35 disposed in the case 25 of the second chamber C2 discharges breath from the second chamber C2 into the housing 3.

Further, in the housing 3, for example, a round housing opening 39 that connects the inside of the housing 3 and the outside thereof is provided in a portion opposing the lower surface of the case 17 of the first chamber C1. The inlet (that is, breath introducing path) 22 which extends downward from the case 17 is disposed so as to pass through the housing opening 39.

The outer diameter of the inlet 22 is less than the inner diameter of the housing opening 39. Therefore, a cylindrical gap 39a is formed between the inner circumferential surface of the housing opening 39 and the outer circumferential surface of the inlet 22.

Therefore, as indicated by an arrow in FIG. 2 and the like, breath (G) from a person is firstly introduced through the inlet 22 into the first chamber C1, passes through the conversion portion 21, and is discharged from the first chamber C1 through the outlet 23 into the gas flow pipe 13.

Next, the breath (G) is introduced from the gas flow pipe 13 through the inlet 33 into the second chamber C2. In the second chamber C2, the breath (G) moves along the detection portion 29a and is discharged through the outlet 35 to the outside of the second chamber C2 (that is, introduced into the housing 3).

The breath (G) discharged to the outside of the second chamber C2 is breath that has been heated in the first chamber C1, and, thereafter, has been further heated in the second chamber C2. Therefore, the temperature of the breath (G) is much higher than the temperature of the breath (G) in a state immediately after the breath (G) has been exhaled from a human body.

Next, the breath having a higher temperature is discharged from the inside of the housing 3 through the housing opening 39 (specifically, through the gap 39a) to the outside of the housing 3. At this time, the temperature of the breath is about 160° C.

At this time, the breath having the higher temperature is discharged to the outside along the outer circumference of the inlet 22. Therefore, heat exchange occurs with the breath having a temperature close to a body temperature and flowing in the inlet 22. That is, the breath having a lower temperature and flowing in the inlet 22 is heated by the breath having a higher temperature and discharged from the housing 3.

A heat exchange portion 41 is formed by a portion in which heat exchange between breath having one temperature and breath having a different temperature occurs, that is, a portion, of the housing opening 39 and the inlet 22, which contacts the breath having a higher temperature in the housing 3.

### [1-3. Principle of Operation of Breath Sensor]

Next, the principle of operation of the breath sensor 1 will be described. This is a known technique as described above, and will be briefly described.

The conversion portion 21 is formed in, for example, the following manner. That is, catalyst powder including a noble metal (for example, platinum) supported on zeolite, y-alumina, or the like, is formed into a slurry and is sintered onto a porous structure or a honeycomb structure through which breath can permeate, thereby forming the conversion portion 21. The conversion portion 21 functions as a porous catalyst. The catalyst allows the first gas component (for example, NO) included in the breath to be converted to the second gas component (for example, NO₂) at a predetermined proportion (that is, a predetermined partial pressure ratio of NO/NO₂) at a predetermined activation temperature (for example, about 300° C.) that is an operation temperature.

Further, the detection portion 29a is structured as a mixed-potential type NOₓ (nitrogen oxide) sensor using a solid electrolyte body and a pair of electrodes disposed on the surface of the solid electrolyte body.

For example, the detection portion 29a may be structured as an element in which a reference electrode formed from Pt and a sensor electrode formed from WO₃ are disposed on a solid electrolyte body formed from YSZ, or structured such that a plurality of the elements each having the solid electrolyte body, the reference electrode, and the sensor electrode, are connected in series.

The detection portion 29a has an electric characteristic which varies with a change in concentration of NOₓ (that is, NO₂) included in the breath (G), at an activation temperature (for example, about 400° C.) that is an operation temperature different from an activation temperature of the catalyst. Specifically, in the detection portion 29a, a voltage is developed between the reference electrode and the sensor electrode which varies with a change in concentration of NO₂. Therefore, a concentration of NO₂ (consequently, a concentration of NO) can be detected based on the difference in potential that is developed between the reference electrode and the sensor electrode.

Further, since the heater 29c is disposed close to the detection portion 29a, the detection portion 29a can be heated to the higher temperature as described above. Meanwhile, since the heater 29c is thermally coupled to the conversion portion 21 through the adhesive layer 31 and the ceramic wiring substrate 9, the temperature of the conversion portion 21 can be made different from the temperature of the detection portion 29a. The temperature of the detection portion 29a is higher than the temperature of the conversion portion 21.

Therefore, in the breath sensor 1, a concentration of NOₓ in the breath (G) can be detected as described below.

As shown in FIG. 2, the breath (G) is firstly introduced into the first chamber C1 through the inlet 22. The conversion portion 21 is heated to a predetermined activation temperature by the heater 29c. Therefore, NO in the breath is converted to NO₂ at a predetermined partial pressure ratio.

After the conversion, the breath (G) is discharged from the first chamber C1 through the outlet 23 into the gas flow pipe 13, and introduced through the inlet 33 into the second chamber C2.

Next, the breath contacts the detection portion 29a in the second chamber C2, whereby a potential difference is developed between the paired electrodes depending on the concentration of NO₂. Therefore, the concentration of NO₂ can be detected based on the potential difference. In this case, NO₂ has been converted from NO at the predetermined partial pressure ratio by the conversion portion 21. Thus, the concentration of NO can be obtained according to the partial pressure ratio.

Further, the breath discharged from the second chamber C2 through the outlet 35 into the housing 3 is discharged to the outside of the housing 3 after heat exchange in the heat exchange portion 41.

### [1-4. Effect]

The breath sensor 1 according to the first embodiment includes the heat exchange portion 41 that allows heat exchange between the breath discharged from the second chamber C2 and the breath introduced into the first chamber C1. Therefore, by breath (that is, discharged breath) discharged from the second chamber C2, the breath (that is, introduced breath) introduced into the first chamber C1 is heated, to increase the temperature of the introduced breath.

Thus, since the temperature of the introduced breath is increased (that is, the introduced breath can be preheated), an effect of reducing power consumption of the heater 29c in heating the conversion portion 21 and the detection portion 29a can be achieved. That is, the heater 29c heats both the conversion portion 21 and the detection portion 29a to a temperature in an operation temperature (that is, activation temperature) range, and, if the introduced breath can be preheated, power consumption for heating to an operation temperature can be reduced.

In particular, in a case where the breath sensor 1 is incorporated in a compact potable device, power consumption of a power supply for heating the heater 29c can be reduced, and the effect thereof is thus significant.

Further, in the first embodiment, the inlet (that is, breath introduction pipe) 22 that passes through the housing opening 39, that is in communication with the inside of the first chamber C1 and the outside of the housing 3, and that allows breath to be introduced from the outside of the housing 3 into the first chamber C1, is provided. Therefore, heat exchange between the introduced breath in the inlet 22, and the discharged breath on the outer circumferential side of the inlet 22 can be efficiently performed. Thus, power consumption of the heater 29c can be further reduced.

### [1-5. Correspondence of Terms]

Correspondence in terms between the present disclosure and the structural features of the first embodiment will next be described.

The first chamber C1, the conversion portion 21, the adjustment unit 5, the second chamber C2, the detection portion 29a, the sensor unit 7, the heater 29c, the gas flow pipe 13, the sensor body portion 37, the housing 3, the heat exchange portion 41, the outlet 35, the housing opening 39, and the inlet 22 in the first embodiment correspond to examples of a first chamber, a conversion portion, an adjustment unit, a second chamber, a detection portion, a sensor unit, a heater, a gas flow path, a sensor body portion, a housing, a heat exchange portion, a chamber opening, a housing opening, and a breath introduction pipe, respectively, in the present disclosure.

### 2. Second Embodiment

Next, a second embodiment will be described.

Description of the same components as in the first embodiment is omitted. The same components as in the first embodiment are denoted by the same reference numerals.

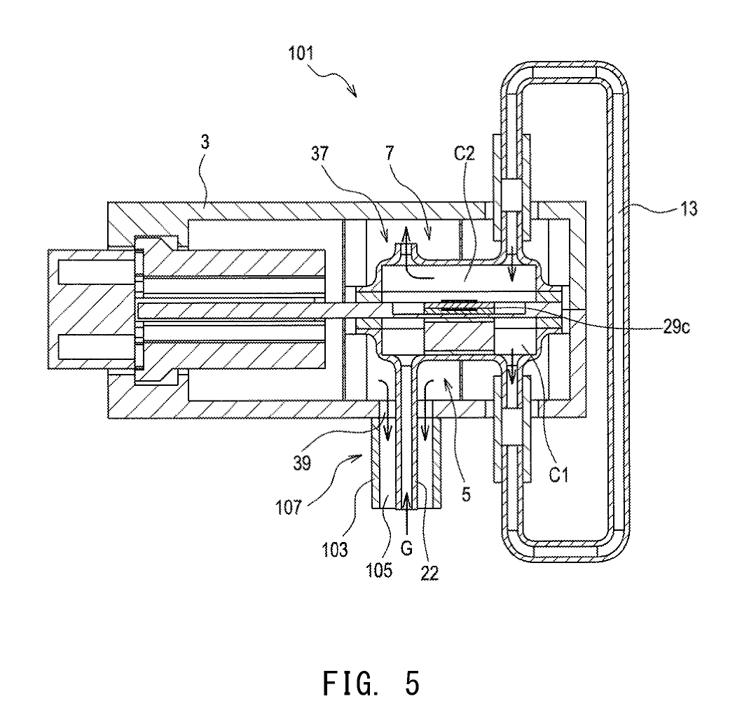

As shown in FIG. 5, a breath sensor 101 according to the second embodiment includes: the sensor body portion 37 having the adjustment unit 5, the sensor unit 7, and the heater 29c disposed in the housing 3; and the like, similarly to the first embodiment. Further, the first chamber C1 and the second chamber C2 are connected to each other by the gas flow pipe 13.

In particular, in the second embodiment, a breath discharge pipe 103 that discharges breath from the inside of the housing 3 to the outside of the housing 3 is provided on the outer surface of the housing 3.

The breath discharge pipe 103 has a through hole 105 that is in communication with the housing opening 39, and is disposed so as to surround the entire circumference of the housing opening 39. Further, the inlet (that is, breath introduction pipe) 22 is disposed so as to pass through the through hole 105 of the breath discharge pipe 103.

In the second embodiment, a heat exchange portion 107 is configured by the breath discharge pipe 103 that surrounds the outer circumferential side portion of the inlet 22, in addition to a portion, of the housing opening 39 and the inlet 22, which contacts breath having a high temperature in the housing 3.

In the second embodiment, an effect similar to that of the first embodiment is achieved. Further, the inlet 22 is disposed so as to pass through the through hole 105 of the breath discharge pipe 103, whereby heat exchange between the introduced breath in the inlet 22 and the discharged breath in the breath discharge pipe 103 can be efficiently performed. Thus, power consumption of the heater 29c can be further reduced.

### 3. Third Embodiment

Next, a third embodiment will be described. Description of the same components as in the first embodiment is omitted. The same components as in the first embodiment are denoted by the same reference numerals.

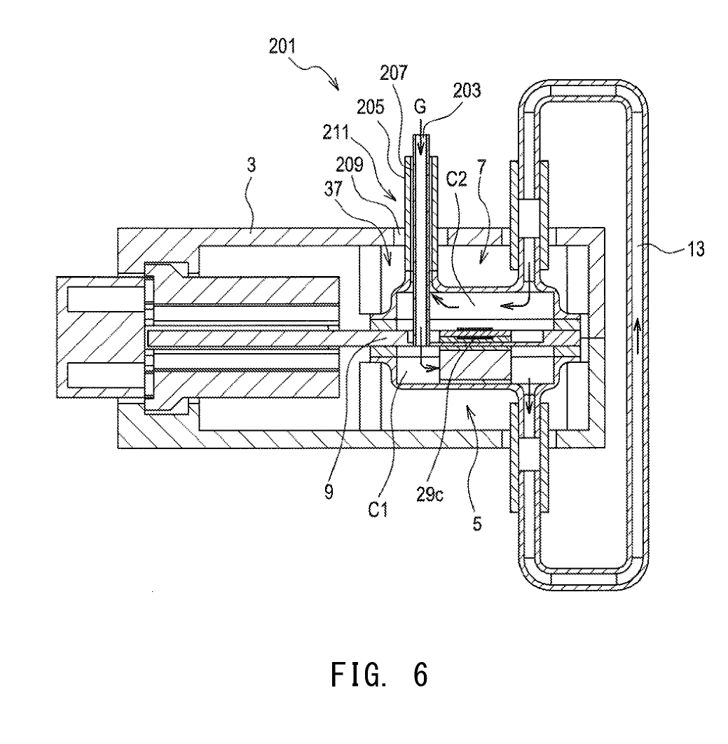

As shown in FIG. 6, a breath sensor 201 of the third embodiment includes: the sensor body portion 37 having the adjustment unit 5, the sensor unit 7, and the heater 29c disposed in the housing 3; and the like, similarly to the first embodiment. Further, the first chamber C1 and the second chamber C2 are connected to each other by the gas flow pipe 13.

In particular, in the third embodiment, an inlet (that is, breath introduction pipe) 203 that extends from the outside of the housing 3 through the second chamber C2 into the first chamber C1, and a breath discharge pipe 205 that extends from the inside of the second chamber C2 to the outside of the housing 3, are provided. Further, the inlet 203 is disposed so as to pass through a through hole 207 of the breath discharge pipe 205. One end portion of the inlet 203 penetrates through the ceramic wiring substrate 9, and is coupled to the ceramic wiring substrate 9 by a not-illustrated sealing member in an airtight manner. Further, the breath flowing in the inlet 203 is introduced into the first chamber C1. The housing 3 has a housing opening 209, and the breath discharge pipe 205 is disposed so as to pass through the housing opening 209.

In the third embodiment, a heat exchange portion 211 is configured by the inlet 203, a portion around the circumference of the inlet 203 in the second chamber C2, the breath discharge pipe 205, and the like.

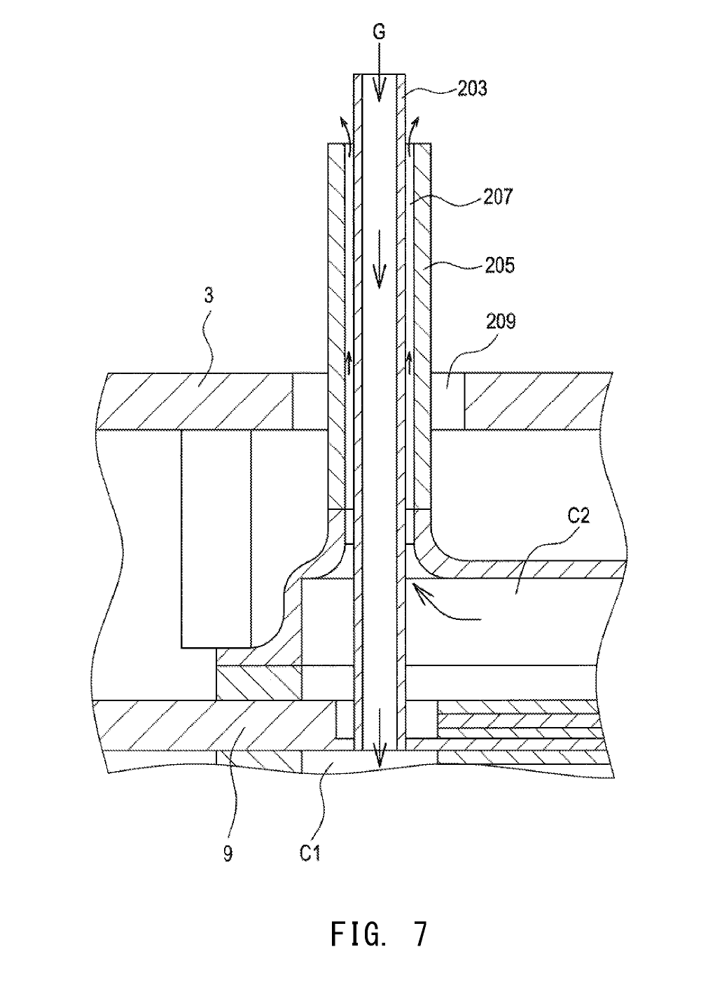

In the third embodiment, an effect similar to that of the first embodiment is achieved. Further, as shown in FIG. 7, the inlet 203 is disposed so as to extend from the outside of the housing 3 through the second chamber C2 into the first chamber C1, and passes through the through hole 207 of the breath discharge pipe 205. Thus, heat exchange between the introduced breath in the inlet 203, breath (that is, breath discharged from the second chamber C2) in the breath discharge pipe 205, and the breath inside the second chamber C2 can be efficiently performed. Thus, power consumption of the heater 29c can be further reduced.

### 4. Other Embodiments

The present disclosure is not limited to the above-described embodiments, and can be implemented in various manners without departing from the scope of the present disclosure.

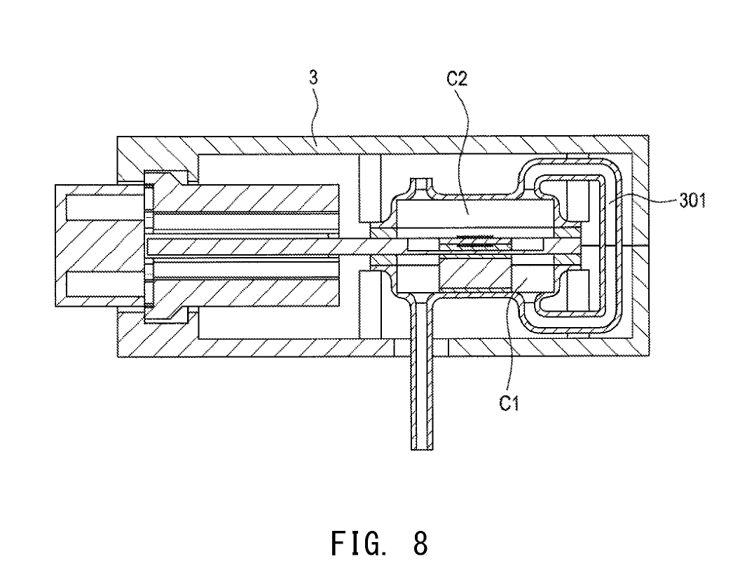

(1) For example, as shown in FIG. 8, a gas flow pipe 301 that connects the first chamber C1 and the second chamber C2 may be disposed inside the housing 3.

(2) In the above-described embodiments, the sensor element portion 29 is coupled with the recess 9a of the ceramic wiring substrate 9 through the adhesive layer 31. However, a heat-insulating sheet formed from a non-woven fabric of an inorganic fiber or the like may be further provided therebetween. Further, when the sensor element portion 29 is mounted to the ceramic wiring substrate 9, the sensor element portion 29 may be mounted on the upper surface of the ceramic wiring substrate 9 without providing the recess 9a.

(3) Further, the conversion portion and the detection portion are not limited to any specific ones, and may be any components, which have the functions described in the present disclosure, other than the components described in the first embodiment.

(4) The function of one component in each embodiment described above may be separated among a plurality of components, while the functions of a plurality of components may be integrated into one component. Further, a part of the structure of the embodiment described above may be omitted. Moreover, at least a part of the structure of the embodiment described above may be, for example, added to or replaced with the structure of another embodiment described above.

The invention has been described in detail with reference to the above embodiments. However, the invention should not be construed as being limited thereto. It should further be apparent to those skilled in the art that various changes in form and detail of the invention as shown and described above may be made. It is intended that such changes be included within the spirit and scope of the claims appended hereto.

What is claimed is:

1. A breath sensor comprising:
an adjustment unit having a first chamber into which breath is introduced, and having a conversion portion that converts, to a second gas component, a first gas component included in the breath that is introduced into the first chamber;
a sensor unit having a second chamber into which the breath that has passed through the adjustment unit is introduced, and having a detection portion having an electric characteristic which varies with a change in concentration of the second gas component;
a single heater configured to heat the conversion portion and the detection portion;
a gas flow path configured to connect the first chamber and the second chamber in a state in which at least a part of the gas flow path extends outside the adjustment unit and outside the sensor unit, wherein
the adjustment unit, the sensor unit, and the heater are integrated into a sensor body portion in a state where the adjustment unit and the heater are thermally coupled to each other, and the sensor unit and the heater are thermally coupled to each other;
a housing which surrounds an outer circumference of the sensor body portion; and
a heat exchange portion that allows for heat exchange between breath discharged from the second chamber and breath introduced into the first chamber and that is provided in at least the housing.

2. The breath sensor as claimed in claim 1, further comprising:
a chamber opening through which breath is discharged from the second chamber into the housing;
a housing opening through which the breath in the housing is discharged to an outside of the housing; and
a breath introduction pipe that passes through the housing opening, connects an inside of the first chamber and the outside of the housing, and allows the breath to be introduced into the first chamber from the outside of the housing.

3. The breath sensor as claimed in claim 2, further comprising:
a breath discharge pipe provided on an outer surface of the housing that allows the breath to be discharged from an inside of the housing to an outside of the housing, wherein
the breath discharge pipe has a through hole that is in communication with the housing opening, and is disposed so as to surround the entirety of a circumference of the housing opening, and the breath introduction pipe is disposed so as to pass through the through hole of the breath discharge pipe.

4. The breath sensor as claimed in claim 1, further comprising:
a breath introduction pipe that extends from an outside of the housing through the second chamber into the first chamber; and
a breath discharge pipe that extends from an inside of the second chamber to an outside of the housing, wherein
the breath introduction pipe is disposed so as to pass through a through hole of the breath discharge pipe.
